SEQUENCE LISTING (4 sequences)

SEQ ID NO: 1 - protein, 5 aa, Artificial
  synthetic ELP pentapeptide
VPGXG

SEQ ID NO: 2 - protein, 758 aa, Artificial sequence
  synthetic ELP
MSKGPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 3 - protein, 568 aa, Artificial sequence
  synthetic ELP
MSKGPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGVGVPGKGVPGVGVPGVGVPGVGVPGVGVPGVGVPGWP

SEQ ID NO: 4 - protein, 688 aa, Artificial sequence
  synthetic ELP
MSKGPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGAGVPGGGVPGAGVPGGGVPGAGVPGGGVPGAGVPGGGVPGAGVPGGGVPGAGVPGGGVPGAGVPGGGVPGAGVPGVGVPGKGVPGVGVPGVGVPGVGVPGVGVPGVGVPGVGVPGFGVPGVGVPGKGVPGVGVPGVGVPGVGVPGVGVPGVGVPGVGVPGFGVPGVGVPGKGVPGVGVPGVGVPGVGVPGVGVPGVGVPGVGVPGFGVPGVGVPGKGVPGVGVPGVGVPGVGVPGVGVPGVGVPGVGVPGFGVPGVGVPGKGVPGVGVPGVGVPGVGVPGVGVPGVGVPGVGVPGFGVPGVGVPGKGVPGVGVPGVGVPGVGVPGVGVPGVGVPGVGVPGFGVPGVGVPGKGVPGVGVPGVGVPGVGVPGVGVPGVGVPGVGVPGFGVPGVGVPGKGVPGVGVPGVGVPGVGVPGVGVPGVGVPGVGVPGFGVPGWP

(19) United States
(12) Patent Application Publication
(10) Pub. No.: US 2011/0110866 A1
(43) Pub. Date: May 12, 2011

(54) Elastin-like polypeptide and gadolinium conjugate for magnetic resonance imaging

(75) Inventors: Ashutosh Chilkoti, Durham, NC (US); Matthew R. Dreher, Rockville, MD (US); Daniel E. Meyer, Rexford, NY (US); Mark W. Dewhirst, Durham, NC (US)

(73) Assignee: Duke University, Durham, NC (US)

(21) Appl. No.: 12/082,271

(22) Filed: Apr. 10, 2008

Related U.S. Application Data
(60) Provisional application No. 60/922,592, filed on Apr. 10, 2007.

(51) Int. Cl. A61K 49/14; C07K 14/00; A61P 35/00; A61P 9/10

(52) U.S. Cl. 424/9.34; 530/400; 428/402

(57) ABSTRACT

A magnetic resonance imaging (MRI) contrast enhancement agent comprising an elastin-like polypeptide (ELP) and one or more paramagnetic metal ions is disclosed. Also disclosed are methods of preparing ELP MRI contrast enhancement agents, formulations comprising ELP MRI contrast enhancement agents, and methods of using ELP MRI contrast enhancement agents to image biological samples and to image and deliver therapeutic agents to targeted sites in vivo. In some embodiments, the ELP MRI agents can be used in methods related to blood volume determination, in magnetic resonance angiography (MRA), and in vascular transport determinations. The ELP MRI contrast agents can also provide information on the expression of various proteins through affinity targeting or enzymatic crosslinking in order to aid in diagnosis and in the spatial definition of pathologic tissue.

### RELATED APPLICATIONS

The presently disclosed subject matter claims the benefit of U.S. Provisional Patent Application Ser. No. 60/922,592, filed Apr. 10, 2007; the disclosure of which is incorporated herein by reference in its entirety.

### GOVERNMENT INTEREST

This presently disclosed subject matter was made with U.S. Government support under Grant Nos. NIBIB R01 EB00188, CA42745, and R24 CA 092656 awarded by the National Institutes of Health. Thus, the U.S. Government has certain rights in the presently disclosed subject matter.

### TECHNICAL FIELD

The presently disclosed subject matter relates to the use of elastin-like polypeptides (ELPs) as magnetic resonance imaging (MRI) contrast enhancement agents. The ELP MRI contrast enhancement agents can be used as targeted MRI agents and to image the delivery of therapeutic agents. In view of the relatively high molecular weight (MW) of the ELP MRI agents, they can also be used as “blood pool” agents in blood volume determination, in magnetic resonance angiography (MRA), and in vascular transport determination (e.g., with DCE-MRI). The ELP MRI contrast agents can provide information on the expression of various proteins through affinity targeting or enzymatic crosslinking in order to aid in the diagnosis and spatial definition of pathologic tissue or pathologic sites within specific tissues or organs.

Table of Abbreviations

| | |
|---|---|
| ° C. | degree Celsius |
| μM | micromolar |
| CDTA | trans-1,2-cyclohexanediamine tetraacetic |
| | acid |
| CMT | critical micelle temperature |
| DCE-MRI | dynamic contrast enhanced magnetic |
| | resonance imaging |
| DMSO | dimethyl sulfoxide |
| DO3A | 1,2,7,10-tetraazacyclododecane-1,4,7- |
| | triacetic acid |
| DOL | degree of labeling |
| DOTA | 1,4,7,10-tetraazacyclododecane-1,4,7,10- |
| | tetraacetic acid |
| DTPA | diethylenetriaminepentaacetate |
| EDTA | ethylenediaminetetraacetate |
| DT | diphtheria toxin |
| ELP | elastin-like polypeptide |
| ELP_{BC} | elastin-like polypeptide block copolymer |
| FDA | Food and Drug Administration |
| Gd | gadolinium |
| GEL | gelonin |
| ICPAES | inductively coupled plasma atomic emission |
| | spectrophotometry |
| ITC | isothiocyanato |
| kDa | kilodaltons |
| kg | kilograms |
| min | minute |
| mM | millimolar |
| mmol | millimole |
| MRA | magnetic resonance angiography |
| MRI | magnetic resonance imaging |
| ms | millisecond |
| MW | molecular weight |
| NHS | N-hydroxysuccinimide ester |
| PBS | phosphate-buffered saline |
| RES | reticulo-endothelial system |
| RF | radio-frequency |
| SDS | sodium dodecyl sulfate |
| T | tesla |
| T1 | longitudinal relaxation |
| T2 | transverse relaxation |
| TETA | tris-(2-aminoethyl)amine |
| TG | transglutaminase |
| Tr | repetition time |
| Tₜ(s) | transition temperature(s) |
| UV-Vis | ultraviolet visible |
| Xaa | guest residue (any amino acid other than |
| | proline) |

Amino Acid Abbreviations, Codes, and Codons

| Amino | 3- | 1- | |
|---|---|---|---|
| Acid | Letter | Letter | Codons |
| Alanine | Ala | A | GCA GCC GCG GCU |
| Arginine | Arg | R | AGA AGG CGA CGC CGG CGU |
| Asparagine | Asn | N | AAC AAU |
| Aspartic Acid | Asp | D | GAC GAU |
| Cysteine | Cys | C | UGC UGU |
| Glutamic acid | Glu | E | GAA GAG |
| Glutamine | Gln | Q | CAA CAG |
| Glycine | Gly | G | GGA GGC GGG GGU |
| Histidine | His | H | CAC CAU |
| Isoleucine | Ile | I | AUA AUC AUU |
| Leucine | Leu | L | UUA UUG CUA CUC CUG CUU |
| Lysine | Lys | K | AAA AAG |
| Methionine | Met | M | AUG |
| Phenylalanine | Phe | F | UUC UUU |
| Proline | Pro | P | CCA CCC CCG CCU |
| Serine | Ser | S | ACG AGU UCA UCC UCG UCU |
| Threonine | Thr | T | ACA ACC ACG ACU |
| Tryptophan | Trp | W | UGG |
| Tyrosine | Tyr | Y | UAC UAU |
| Valine | Val | V | GUA GUC GUG GUU |

### BACKGROUND

Magnetic resonance imaging (MRI) is a widely used and powerful diagnostic imaging technique that uses radiofrequency (RF) energy in the presence of a magnetic field to extract information about atomic nuclei having the appropriate electronic spin characteristics (typically hydrogen in medical imaging due to its relative abundance within the body). See Rudin and Weissleder, Nat. Rev. Drug Discov., 2(2), 123-131 (2003). When these nuclei are placed in a strong magnetic field, they align either parallel or anti-parallel with the magnetic field and are thus in an overall low energy state. Next, the nuclei are exposed to a radiofrequency pulse that aligns the nuclei in a plane perpendicular to the magnetic field, which places them in a high energy state. Following the radiofrequency excitation, the high energy nuclei relax and realign with the main magnetic field. As the nuclei relax they emit RF energy that is detected and converted into a spatially encoded signal. The rate of relaxation is dependent upon the environment of the nuclei. The realignment of magnetic spins in the direction of the magnetic field is called spin-lattice, longitudinal relaxation, or T1. The dephasing of the nuclear spins after the radiofrequency pulse is called spin-spin relaxation, transverse relaxation or T2.

Image contrast is generated by selecting sequences of RF pulses that weight the signal intensity obtained within an image. These pulse sequences are often chosen to weight image contrast by either the T1 or T2 relaxation rates, which inherently vary depending on tissue properties. Additional contrast may be obtained by administering an exogenous agent to the patient such as paramagnetic or superparamagnetic compounds. These agents create a local net magnetic moment in the presence of the external magnetic field and can increase the relaxation rates of nearby hydrogen nuclei, thereby increasing the signal intensity (i.e., contrast) of the image.

The most widely used FDA approved MRI contrast agents are MAGNEVIST® (gadopentetate dimeglumine, molecular weight (MW)=938; available from Bayer HealthCare Pharmaceuticals Inc., Wayne, N.J., United States of America) and OMNISCAN™ (gadodiamide, MW=574; available from GE Healthcare, Princeton, N.J., United States of America). These FDA approved contrast agents chelate the gadolinium ion. Free gadolinium is toxic. The LD₅₀ of free gadolinium is approximately 0.5 mmol/kg, while the LD₅₀ of chelated gadolinium is about 20 times higher, about 10 mmol/kg. Free gadolinium is also strongly retained in the body with only 2% of the injected dose cleared after 7 days. See Weinmann, et al., AJR Am. J. Roentgenol., 142(3), 619-624 (1984). Unchelated gadolinium tends to accumulate largely in the liver and bone with lesser accumulation in the spleen and lung. See Franano, et al., Magn. Reson. Imaging, 13(2), 201-214 (1995); Gibby et al., Invest. Radiol., 39(3), 138-142 (2004); and Wedeking, et al., Magn. Reson. Imaging, 10(4), 641-648 (1992). To avoid potential liberation of the gadolinium from its chelator, the toxicity concerns of gadolinium require that even the chelated agent be rapidly cleared through the kidney to minimize residence time within the body and to limit long term exposure to gadolinium. In particular, the distribution and elimination half-lives for MAGNEVIST° are 12 min and 96 min, while those for OMNISCAN™ are 3.7 min and 77.8 min, respectively. See Berlex, MAGNEVIST® Product Information Sheet, (2006); and GE Healthcare, OMNISCAN™ Product Information Sheet, (2005). The rapid renal elimination of these agents is due to their low MW (<10 kDa); however, these low MW agents also rapidly extravasate in most tissues and therefore do not provide a good estimate of tissue blood volumes and require rapid image acquisition for dynamic contrast enhanced MRI (DCE-MRI).

Recently, there has been increased interest in high MW, “blood pool” contrast agents (>10 kDa) to better define blood volume and potentially better determine parameters such as k^{trans} that describe vascular transport. See Dafni, et al., Cancer Res., 62(22), 6731-6739 (2002); Dafni, et al., NMR Biomed., 15(2), 120-131 (2002); Israely, et al., Magn, Reson. Med., 52(4), 741-750 (2004); Kobayashi, et al., Clin. Cancer Res., 10(22), 7712-7720 (2004); Wang, et al., Pharm. Res., 21(10), 1741-1749 (2004); and Yordanov, et al., J. Mater, Chem., 13(7), 1523-1525 (2003). Despite the potential utility for such high MW contrast agents, there are currently no clinically approved high MW agents because the longer half-lives and retention of these agents increase toxicity concerns.

Accordingly, there is a need for high MW contrast agents for use in MRI that are biocompatible and have long plasma half-lives. In particular, there is a need for high MW contrast agents for use in blood volume determination, magnetic resonance angiography (MRA), and in vascular transport determination.

### SUMMARY

In some embodiments, the presently disclosed subject matter provides a contrast agent comprising an elastin-like polypeptide (ELP) and one or more paramagnetic metal ions.

In some embodiments, the paramagnetic metal ion is selected from the group consisting of a transition element, a lanthanide element, and an actinide element. In some embodiments, the paramagnetic metal ion is selected from the group consisting of Gd(III), Mn(II), Cu(II), Cr(III), Fe(II), Fe(III), Co(II), Er(II), Ni(II), Eu(III) and Dy(III). In some embodiments, the paramagnetic metal ion is Gd(III).

In some embodiments the contrast enhancement agent further comprises one or more bifunctional chelators. In some embodiments, the one or more bifunctional chelators each comprise a metal chelator selected from the group consisting of diethylenetriaminepentaacetate (DTPA), 1,4,7,10-tetraazacyclododecane-1,4,7,10-tetraacetic acid (DOTA), 1,2,7,10-tetraazacyclododecane-1,4,7-triacetic acid (DO3A), trans-1,2-cyclohexanediamine tetraacetic acid (CDTA), ethylenediaminetetraacetic acid (EDTA), and tris-(2-aminoethyl)amine (TETA). In some embodiments, each metal chelator is DOTA.

In some embodiments, the one or more bifunctional chelators are each bonded to the ELP via a covalent linkage. In some embodiments, each covalent linkage is independently selected from an amide and a thiourea. In some embodiments, the one or more bifunctional chelators are each bonded to the ELP via the amino group of a lysine residue within the ELP backbone or the amino group of the ELP N-terminal residue.

In some embodiments, the ELP comprises one or more lysine residues. In some embodiments, the ELP comprises at least 9 lysine residues. In some embodiments, the ELP comprises at least 17 lysine residues.

In some embodiments, the ELP has a molecular weight greater than about 10 kDa. In some embodiments, the ELP has a molecular weight greater than about 40 kDa.

In some embodiments, the ELP has an amino acid sequence comprising one of the group consisting of SEQ ID NO: 2, SEQ ID NO: 3, and SEQ ID NO: 4. In some embodiments, the ELP has an amino acid sequence comprising SE(} ID NO: 3.

In some embodiments, the contrast enhancement agent comprises a plurality of paramagnetic metal ions. In some embodiments, the contrast enhancement agent comprises at least 10 paramagnetic metal ions. In some embodiments, the contrast enhancement agent comprises at least 18 paramagentic metal ions.

In some embodiments, the contrast enhancement agent forms a micelle. In some embodiments, the contrast enhancement agent has a diameter greater than 40 nm.

In some embodiments, the contrast enhancement agent has a relaxivity of 7.0 mM⁻¹ s⁻¹ or greater at 2T based on the concentration of paramagnetic ion.

In some embodiments, the contrast enhancement agent further comprises one or more targeting agents. In some embodiments, the targeting agent is selected from an antibody, an antibody fragment, or a peptide (e.g., RGD or NGR sequence). In some embodiments, the targeting agent comprises a small molecule (e.g., folate), a peptidomimetic, or a nucleotide-derived aptamer.

In some embodiments, the contrast enhancement agent further comprises an enzymatically recognized reaction site. In some embodiments, the enzymatically recognized reaction site is cross-linkable via enzymatic catalysis to one of the group consisting of another contrast agent, a cell, and a tissue. In some embodiments, the enzymatically recognized reaction site is hydrolyzable via enzymatic catalysis.

In some embodiments, the contrast enhancement agent further comprises a therapeutic agent. In some embodiments, the therapeutic agent is a neoplastic agent.

In some embodiments, the contrast enhancement agent further comprises an optical imaging moiety.

In some embodiments, the presently disclosed subject matter provides a formulation comprising a contrast enhancement agent, said contrast enhancement agent comprising an ELP and a paramagnetic metal ion, and a pharmaceutically acceptable carrier.

In some embodiments, the presently disclosed subject matter provides a formulation comprising a contrast enhancement agent, said contrast enhancement agent comprising an ELP and a paramagnetic metal ion, and an optical imaging moiety.

In some embodiments, the presently disclosed subject matter provides a method of generating a visible image of a biological sample, the method comprising: contacting the biological sample with a contrast enhancement agent, the contrast enhancement agent comprising an elastin-like peptide (ELP) and one or more paramagnetic metal ions; and rendering a magnetic resonance image of the sample.

In some embodiments, the sample is one of a cell, a tissue, an organ and a subject. In some embodiments, the subject is a human.

In some embodiments, generating a visible image of the biological sample further indicates the presence of a disease state. In some embodiments, the disease state is cancer or atherosclerosis.

In some embodiments, generating a visible image of the biological sample further indicates the delivery of a therapeutic agent.

In some embodiments, contacting the biological sample further comprises targeting the biological sample with a targeting agent associated with the contrast enhancement agent. In some embodiments, contacting the biological sample further comprises cross-linking the contrast agent to the biological sample via an enzymatically catalyzed reaction.

In some embodiments, generating the visible image is part of a procedure selected from the group consisting of blood volume determination, magnetic resonance angiography (MRA), and vascular transport determination.

In some embodiments, a visible image is generated with MRI to identify the location of pathologic tissue, such as cancer or arterial plaque, to guide surgical resection of the tissue. An optical imaging moiety associated with the contrast agent can then guide the surgeon interoperatively.

In some embodiments, the presently disclosed subject matter provides a method of preparing an ELP contrast enhancement agent, the method comprising: providing an ELP, the ELP comprises at least one primary amine group; providing a bifunctional chelator group, wherein the bifunctional chelator group comprises a group that can interact with the amine; contacting the ELP and the bifunctional chelator group such that the bifunctional chelator group interacts with the amine to form an ELP-chelator conjugate; providing a paramagnetic metal ion; and contacting the ELP-chelator conjugate with the paramagnetic metal ion to chelate the metal ion with the ELP-chelator, thereby preparing an ELP contrast enhancement agent.

In some embodiments, the bifunctional chelator group forms a covalent bond with the amine group on the ELP. In some embodiments, the covalent bond is one of an amide and a thiourea.

It is an object of the presently disclosed subject matter to provide a contrast enhancement agent comprising an ELP peptide and a paramagnetic metal ion.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic drawing showing three different elastin-like peptide (ELP) gadolinium (Gd) conjugates. ELP1-150 (SEQ ID NO: 2) has one lysine placed near its N-terminus to conjugate gadolinium at just one end of the ELP biopolymer. ELP5-112 (SEQ ID NO: 3) has 17 lysine residues placed throughout the ELP's backbone to increase gadolinium loading on the ELP. ELP2-64,12-72 (SEQ ID NO: 4) has one lysine residue at the N-terminus and 8 lysine residues on the C-terminal block of the ELP to demonstrate that gadolinium may be placed at specific sites or within specified regions of the biopolymer.

FIG. 2 is a conjugation scheme for a diethylenetriaminepentaacetate-isothiocyanoato (DTPA-ITC) group with ELP. The isothiocyanato (ITC) group on the DTPA-ITC reacts with amines on the ELP to produce a thiourea bond.

FIG. 3 is a conjugation scheme for a 1,4,7,10-tetraazacyclododecane-1,4,7,10-tetraacetic acid-N-hydroxysuccinimide (DOTA-NHS) with ELP. The N-hydroxysuccinimide ester group on the DOTA-NHS reacts with amines on the ELP to produce an amide bond.

FIG. 4 is a turbidity profile for ELP-Gd conjugates prepared using DOTA-NHS and for the parent ELPs. Turbidity profiles were obtained by monitoring optical density (OD) at 350 nm in PBS from an upward thermal ramp (1° C./min). The increase in OD indicates formation of ELP aggregates in solution. The sharpness of the phase transition and Tₜ can be obtained from dOD/dT as shown in the top portion of the figure. Each ELP is identified by a unique color. ELP5-112 (SEQ ID NO: 3) is indicated by the green lines. ELP2-64,12-72 (SEQ ID NO: 4) is indicated by the blue lines. ELP1-150 (SEQ ID NO: 2) is indicated by the gold lines. The solid lines are the parent ELPs and the dashed lines are the gadolinium conjugates.

FIG. 5 is a magnetic resonance imaging (MRI) image of ELP-Gd conjugates. ELP5-112 (SEQ ID NO: 3) conjugates are shown on the left side of the image. ELP2-64,12-72 (SEQ ID NO: 4) conjugates are shown on the right side of the image. The concentration of gadolinium increases from left to right. On the top row, the gadolinium concentration is 62.5 μM, 125 μM, and 250 μM. On the bottom row, the gadolinium concentration is 500 μM, 1000 μM, and 2000 μM.

FIG. 6 is a plot of signal versus repetition time (Tr) for the conjugate of ELP5-112 (SEQ ID NO: 3) and gadolinium (Gd). Images were acquired with a spin echo sequence and signal intensities were obtained by arbitrarily defined regions of interest. The solid line is a fit to the data (solid symbols) with equation 1. Concentration of the conjugate is indicated by the color of the data points: red for 62.5 μM, orange for 125 μM, yellow for 250 μM, green for 500 μM, light blue for 1000 μM, and dark blue for 2000 μM.

FIG. 7 is a plot of the relaxivity of the ELP5-112 (SEQ ID NO: 3)-Gd conjugate. Relaxivity is expressed in terms of Gd concentration.

FIG. 8 is a plot of relaxivity versus temperature for the ELP1-150 (SEQ ID NO: 2)-Gd conjugate and for Gd-DTPA. Relaxivity is expressed in terms of Gd concentration.

FIG. 9A is a magnetic resonance image of a BALB/c nude mouse injected with Gd-DTPA (0.3 mmol Gd/kg).

FIG. 9B is a magnetic resonance image of a BALB/c nude mouse injected with an ELP-Gd conjugate (0.03 mmol Gd/kg). The ELP-Gd conjugate was prepared using ELP5-112 (SEQ ID NO: 3) conjugated to DOTA.

FIG. 10 is a graph comparing the vascular contrast enhancement observed for ELP-Gd and Gd-DTPA. Error bars: ANOVA p<0.0001, *p,0.05(Tukey), n=3.

### BRIEF DESCRIPTION OF THE SEQUENCE LISTING

SEQ ID NO: 1 is the amino acid sequence of the base unit of the ELP peptide, Val-Pro-Gly-Xaa-Gly, where the “guest residue” Xaa is any amino acid except Pro.

SEQ ID NO: 2 is the amino acid sequence of ELP1-150, an ELP peptide comprising one lysine residue and having a MW of 59.4 kDa, which can be employed in the preparation of gadolinium conjugates for use as MRI contrast enhancement agents.

SEQ ID NO: 3 is the amino acid sequence of ELP5-112, an ELP peptide comprising 17 lysine residues and having a MW of 47.1 kDa, which can be employed in the preparation of gadolinium conjugates for use as MRI contrast enhancement agents.

SEQ ID NO: 4 is the amino acid sequence of ELP2-64,12-72, an ELP block copolymer (ELP_{BC}) comprising 9 lysine residues and having a MW of 55.1 kDa, which can be employed in the preparation of gadolinium conjugates for use as MRI contrast enhancement agents.

### DETAILED DESCRIPTION

The presently disclosed subject matter generally relates generally to compositions and methods for magnetic resonance imaging (MRI). In one embodiment, the presently disclosed subject matter relates to a contrast enhancement agent comprising an elastin-like polypeptide (ELP) and a paramagnetic metal ion.

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

All publications, patent applications, patents, and other references mentioned herein are incorporated by reference in their entirety.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described:

Following long-standing patent law convention, the terms “a”, “an”, and “the” refer to “one or more” when used in this application, including the claims. Thus, for example, reference to “a metal ion” includes a plurality of such metal ions, and so forth.

Unless otherwise indicated, all numbers expressing quantities of reagents, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of mass, weight, concentration or percentage is meant to encompass variations of in one example ±20% or ±10%, in another example ±5%, in another example ±1%, and in still another example ±0.1% from the specified amount, as such variations are appropriate to perform the disclosed methods.

As used herein, the terms “amino acid” and “amino acid residue” are used interchangeably and refer to any of the twenty naturally occurring amino acids. An amino acid is formed upon chemical digestion (hydrolysis) of a polypeptide at its peptide linkages. The amino acid residues described herein are in some embodiments in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxy terminus of a polypeptide. In keeping with standard polypeptide nomenclature, abbreviations for amino acid residues are shown in tabular form presented hereinabove.

It is noted that all amino acid residue sequences represented herein by formulae have a left-to-right orientation in the conventional direction of amino terminus to carboxy terminus. In addition, the phrases “amino acid” and “amino acid residue” are broadly defined to include modified and unusual amino acids.

Furthermore, it is noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

The term “nucleic acid molecule” refers to deoxyribonucleotides or ribonucleotides and polymers thereof in either single- or double-stranded form. Unless specifically limited, the term encompasses nucleic acids containing known analogues of natural nucleotides that have similar properties as the reference natural nucleic acid. Unless otherwise indicated, a particular nucleotide sequence also implicitly encompasses conservatively modified variants thereof (e.g., degenerate codon substitutions), complementary sequences, subsequences, elongated sequences, as well as the sequence explicitly indicated. The terms “nucleic acid molecule” or “nucleotide sequence” can also be used in place of “gene”, “cDNA”, or “mRNA”. Nucleic acids can be derived from any source, including any organism. Additionally, nucleic acids can be synthesized using techniques known in the art.

The term “isolated”, as used in the context of a nucleic acid molecule, indicates that the nucleic acid molecule exists apart from its native environment and is not a product of nature. An isolated DNA molecule can exist in a purified form or can exist in a non-native environment such as a recombinant host cell.

The term “isolated”, as used in the context of a polypeptide, indicates that the polypeptide exists apart from its native environment and is nota product of nature. An isolated polypeptide can exist in a purified form or can exist in a non-native environment such as, for example, in a recombinant host cell.

The term “gene” refers broadly to any segment of DNA associated with a biological function. A gene encompasses sequences including, but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “gene expression” generally refers to the cellular processes by which a biologically active polypeptide is produced from a DNA sequence.

The term “operatively linked”, as used herein, refers to a promoter region that is connected to a nucleotide sequence in such a way that the transcription of that nucleotide sequence is controlled and regulated by that promoter region. Similarly, a nucleotide sequence is said to be under the “transcriptional control” of a promoter to which it is operatively linked. Techniques for operatively linking a promoter region to a nucleotide sequence are known in the art.

The terms “heterologous gene”, “heterologous DNA sequence”, “heterologous nucleotide sequence”, “exogenous nucleic acid molecule”, or “exogenous DNA segment”, as used herein, refer to a sequence that originates from a source foreign to an intended host cell or, if from the same source, is modified from its original form. Thus, a heterologous gene in a host cell includes a gene that is endogenous to the particular host cell but has been modified, for example by mutagenesis or by isolation from native transcriptional regulatory sequences. The terms also include non-naturally occurring multiple copies of a naturally occurring nucleotide sequence. Thus, the terms refer to a DNA segment that is foreign or heterologous to the cell, or homologous to the cell but in a position within the host cell nucleic acid wherein the element is not ordinarily found.

As used herein, the term “expression construct” refers to a DNA sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression.

Nucleic acids used to prepare the polypeptides of the presently disclosed subject matter can be cloned, synthesized, recombinantly altered, mutagenized, or combinations thereof. Standard recombinant DNA and molecular cloning techniques used to isolate nucleic acids are known in the art. Exemplary, non-limiting methods are described by Silhavy et al., 1984 (Experiments with Gene Fusions. Cold Spring Harbor Laboratory, Cold Spring Harbor, N.Y., United States of America); Ausubel et al., 1989 (Current Protocols in Molecular Biology. Wiley, New York, N.Y., United States of America); Glover and Hames, 1995 (DNA Cloning: A Practical Approach. Oxford; IRL Press at Oxford University Press, New York, N.Y., United States of America); and Sambrook and Russell, 2001 (Molecular Cloning: A Laboratory Manual. Cold Spring Harbor Laboratory Press, Cold Spring Harbor, N.Y., United States of America). Site-specific mutagenesis to create base pair changes, deletions, or small insertions is also known in the art.

As used herein, the term “polypeptide” means any polymer comprising any of the 20 protein amino acids, or amino acid analogs, regardless of its size or function. Although “protein” is often used in reference to relatively large polypeptides and “peptide” is often used in reference to small polypeptides, usage of these terms in the art overlaps and varies. The term “polypeptide” as used herein refers to peptides, polypeptides and proteins, unless otherwise noted. Thus, as used herein, the terms “protein”, “polypeptide” and “peptide” are used interchangeably.

The polypeptides employed in accordance with the presently disclosed subject matter include, but are not limited to a therapeutic polypeptide as defined herein below; a polypeptide substantially identical to a therapeutic polypeptide as defined herein below; a polypeptide fragment of a therapeutic polypeptide as defined herein below (in one embodiment biologically functional fragments), fusion proteins comprising a therapeutic polypeptide as defined herein below, biologically functional analogs thereof, and polypeptides that cross-react with an antibody that specifically recognizes a therapeutic polypeptide as defined herein below. The polypeptides employed in accordance with the presently disclosed subject matter include, but are not limited to isolated polypeptides, polypeptide fragments, fusion proteins comprising the disclosed amino acid sequences, biologically functional analogs, and polypeptides that cross-react with an antibody that specifically recognizes a disclosed polypeptide.

The presently disclosed subject matter also encompasses recombinant production of the disclosed polypeptides. Briefly, a nucleic acid sequence encoding a polypeptide is cloned into an expression construct and the expression construct is introduced into a host organism, where it is recombinantly produced.

As used herein, the term “paramagnetic metal ion” refers to a metal ion that is magnetized parallel or antiparallel to a magnetic field to an extent proportional to the field. Generally, paramagnetic metal ions are metal ions that have unpaired electrons. Paramagnetic metal ions can be selected from the group consisting of transition and inner transition elements, including, but not limited to, scandium, titanium, vanadium, chromium, cobalt, nickel, copper, molybdenum, ruthenium, cerium, praseodymium, neodymium, promethium, samarium, europium, terbium, holmium, erbium, thulium, and ytterbium. In some embodiments, the paramagnetic metal ions can be selected from the group consisting of gadolinium III (i.e., Gd⁺³ or Gd(III)); manganese II (i.e., Mn⁺² or Mn(II)); copper II (i.e., Cu⁺² or Cu(II)); chromium III (i.e., Cr⁺³ or Cr(III)); iron II (i.e., Fe⁺² or Fe(II)); iron III (i.e., Fe⁺³ or Fe(III)); cobalt II (i.e., Co⁺² or Co(II)); erbium II (i.e., Er⁺² or &Op), nickel II (i.e., Ni⁺² or Ni(II)); europium III (i.e., Eu⁺³ or Eu(III)); yttrium III (i.e., Yt⁺³ or Yt(III)); and dysprosium III (i.e., Dy⁺³ or Dy(III)). In some embodiments, the paramagnetic ion is the lanthanide atom Gd(III), due to its high magnetic moment, symmetric electronic ground state, and its current approval for diagnostic use in humans.

The term “bonding” or “bonded” and variations thereof can refer to either covalent or non-covalent bonding. In some cases the term bonding refers to bonding via a coordinate bond.

As used herein the term “conjugate” refers to a species that comprises the interaction or association of one or more subspecies. The interaction of individual subspecies can involve covalent bonding, non-covalent bonding (i.e., hydrogen bonding, van der Waals interactions, etc.) or coordinate bonding, such as the chelation of a metal ion. The subspecies can include any combination of small molecules, polypeptides, proteins, oligonucleotides, and ions. In some embodiments, the term “conjugate” refers to a species that comprises an ELP and a paramagnetic metal ion. In some embodiments, a “conjugate” refers to a species that comprises an ELP, one or more bifunctional linker moieties, and one or more paramagnetic metal ions.

The term “coordination” refers to an interaction in which one multi-electron pair donor coordinately bonds, i.e., is “coordinated,” to one metal ion.

The term “coordinate bond” refers to an interaction between an electron pair donor and a coordination site on a metal ion resulting in an attractive force between the electron pair donor and the metal ion. The use of this term is not intended to be limiting, in so much as certain coordinate bonds also can be classified as have more or less covalent character (if not entirely covalent character) depending on the characteristics of the metal ion and the electron pair donor.

The term “coordination site” refers to a point on a metal ion that can accept an electron pair donated, for example, by a chelating agent.

The terms “chelating agent” and “chelator” refer to a molecule or molecular ion having two or more unshared electron pairs available for donation to a metal ion. In some embodiments, the metal ion is coordinated by two or more electron pairs to the chelating agent. The terms “bidentate chelating agent,” “tridentate chelating agent,” “tetradentate chelating agent,” and “pentadentate chelating agent” refer to chelating agents having two, three, four, and five electron pairs, respectively, available for simultaneous donation to a metal ion coordinated by the chelating agent. In some embodiments, the electron pairs of a chelating agent form coordinate bonds with a single metal ion. In some embodiments, the electron pairs of a chelating agent form coordinate bonds with more than one metal ion, with a variety of binding modes being possible.

The term “bifunctional chelator” as used herein refers to a moiety that comprises a chelator that can chelate a metal ion and a second group that is capable of bonding to another species, such as a second ion, a small molecule or a peptide or protein. In some embodiments, the term bifunctional chelator refers to a moiety suitable for attachment to both a protein or peptide and a metal ion. Thus, in addition to having a metal binding moiety (i.e., a chelator), these compounds also possess reactive functional groups useful for attachment to proteins or peptides. Suitable peptide-reactive functional groups are known in the art. Examples of these groups are isothiocynato, bromoacetamido, diazo, N-hydroxysuccinimide esters and anhydrides. These groups can be incorporated into known chelating agents. The chelator can comprise a bivalent linker group located between the chelating moiety and the reactive functional group. Examples of linkers include, but are not limited to, alkylene groups (i.e., —(CH₂)ₙ—), arylene groups (e.g., phenylene), or heteroatom-comprising oligomeric groups such as polyethylene glycol (i.e. —(OCH₂CH₂O)ₙ—) and polypropylene glycol (i.e., —(OCH(CH₃)CH₂O)ₙ—). Alternatively, the chelator can also comprise a peptide-based linker group.

The terms “contrast agent” and “contrast enhancement agent” as used herein describe a substance that improves the visibility of structures during a radiographic study.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “amino” refers to the —NH₂ group.

### II. Elastin-Like Polypeptide Gadolinium Conjugates

II.A. Elastin-Like Polypeptides

Elastin-like polypeptides (ELPs) are a class of temperature responsive biopolymers that are derived from a structural motif found in mammalian elastin. See Gray et al., Nature, 246(5434), 461-466 (1973); and Tatham, Trends Biochem. Sci., 25(11), 567-571 (2000). This family of polypeptides comprises polymers of the pentapeptide Val-Pro-Gly-Xaa-Gly (SEQ ID NO: 1), where the “guest residue” Xaa is any amino acid except Pro. Xaa can be the same or different in each repeat of SEQ ID NO: 1. In some embodiments, an ELP comprises at least ten, twenty, thirty, forty, fifty, sixty, or more repeats of Val-Pro-Gly-Xaa-Gly (SEQ ID NO: 1). As used herein, the terms “elastin-like peptide”, “elastin-like polypeptide”, and “elastin-like protein” are used interchangeably and refer to polypeptides comprising polymers of the pentapeptide Val-Pro-Gly-Xaa-Gly (SEQ ID NO: 1).

ELPs undergo an inverse temperature phase transition, also known as a lower critical solution temperature transition, in aqueous solution in response to an increase in solution temperatures. See Li, et al., J. Am. Chem. Soc., 123(48), 11991-11998 (2001); Urry, Prog. Biophys. Mol. Biol., 57(1), 23-57 (1992); and Urry, J. Phys. Chem. B, 101(51), 11007-11028 (1997). Below their transition temperature (Tₜ) they are soluble in aqueous solution. Above their Tₜ, however, ELPs undergo a sharp phase transition (˜2° C.) during which they hydrophobically collapse and aggregate. This phase transition is fully reversible, so that the aggregated ELP dissolves in aqueous solution once the temperature is decreased below the Tₜ. As a result, an ELP is also referred to herein as a “thermally responsive polypeptide” and/or “temperature-sensitive polypeptide”.

As used herein, the term “transition temperature” or “Tₜ” refers to the temperature above which a polymer (for example, an ELP) that undergoes an inverse temperature transition is insoluble in an aqueous system (e.g., water, physiological saline solution, blood, or serum), and below which such a polymer is soluble in the aqueous system. Representative Tₜs for in vitro applications include 0° C., 10° C., 15° C., 20° C. to 60° C., and 60° C. to 100° C., including all temperatures in between. For in vivo applications, representative Tₜs include 35° C. to 65° C. inclusive, including, but not limited to 35-40° C., 40-45° C., 45-50° C. 50-55° C., and 55-60° C.

One of skill in the art can employ an ELP having a certain Tₜ based upon such parameters as what temperatures the ELP is to be exposed to and whether it would be desirable for the ELP to remain soluble or become insoluble under specific conditions. In some embodiments, the Tₜ can be tuned by adjusting one or more of the guest residues (Xaa of SEQ ID NO: 1), the MW (or length of the ELP), and the ELP concentration. See Meyer and Chilkoti. Biomacromolecules, 3(2), 357-367 (2002); and Meyer and Chilkoti. Biomacromolecules, 5(3), 846-851 (2004). For example, generally, as the hydrophobicity of the guest residue increases, the Tₜ decreases. Thus, for ELPs that comprise polymers of SEQ ID NO: 1, as the mole fraction of guest residues that are hydrophobic increases, the Tₜ of the ELP decreases. As such, ELPs can be synthesized with different Tₜs based upon the mole fraction of different residues chosen as the guest residue. The relative hydrophobicities and hydrophilities of the naturally occurring amino acids are known, as well as the general effect on Tₜ that can be expected when a given amino acid is present as the guest residue. The hierarchy of guest residues from most hydrophobic (that is, having the largest lowering effect on Tₜ) to least hydrophobic is Trp-Tyr-Phe-Leu-Ile-Met-Val-Cys-Ala-Thr-Asn-Ser-Gly-Arg-Gln-Lys. See Urry, et al., J. Am. Chem. Soc., 113(11), 4346-4344 (1991); and Urry, et al., J. Phys Chem. B., 101(51), 11007-11028 (1997).

Additionally, a longer ELP will have a lower Tₜ than a shorter ELP with the same mole fraction of various guest residues. Thus, another way to influence the Tₜ of a given ELP is to lengthen or shorten it. For a given mole fraction of individual guest residues, the Tₜ can be varied over 20° C. or more depending on the length of the ELP. An example of this effect is described by Meyer and Chilkoti (Biomacromolecules, 3(2), 357-367 (2002)), where for an ELP with only Val, Ala, and Gly guest residues in a ratio of 5:2:3, respectively, a 60 amino acid ELP had a Tₜ of about 62° C. (25 μM in PBS), a 90 amino acid ELP has a Tₜ of about 50° C., a 150 amino acid ELP has a Tₜ of about 42° C., a 240 amino acid ELP has a Tₜ of about 38° C., and a 330 amino acid ELP has a Tₜ of about 36° C. In the same study, an ELP with only Val, Ala, and Gly guest residues in a ratio of 1:8:7, respectively, a 128 amino acid ELP has a Tₜ of about 77° C. (25 μM in PBS), a 160 amino acid ELP has a Tₜ of about 71° C., a 256 amino acid ELP has a Tₜ of about 63° C., and a 320 amino acid ELP has a Tₜ of about 60° C. Thus, by manipulating the mole fraction of the guest residue and the length of the ELP polypeptide, ELPs with Tₜs between about 20° C. and 80° C. can be designed.

In some embodiments, the ELP can comprise an ELP block copolymer (ELP_{BC}). The ELP_{BC}s can have a linear AB diblock architecture, formed by seamlessly fusing an N-terminal ELP gene with a high Tₜ (Tₜ>90° C., termed ELP2) to a C-terminal ELP gene that has a much lower Tₜ (Tₜ≅40° C., termed ELP12). These ELP_{BC}s are highly soluble at a solution temperature below the Tₜ of both ELP blocks. However, upon an increase in solution temperature the ELP_{BC}s often self-assemble into a spherical micelle when the low Tₜ block undergoes its inverse temperature phase transition. The temperature at which the ELP_{BC} forms a micelle is defined as the critical micelle temperature (CMT). The notation for the ELP_{BC}s consists of an N-terminal ELP gene followed by its number of pentapeptides, then a C-terminal ELP gene and its corresponding number of pentapeptides. For example, ELP2-64,12-72 is an ELP_{BC} with 64 pentapeptides of an ELP2 gene at the N-terminus followed by 72 pentapeptides of ELP12 at the C-terminus.

II.B. ELP MRI Contrast Agents

Numerous macromolecular contrast agents have been proposed including dendrimers (see Kobayashi, et al., Clin. Cancer Res., 10(22), 7712-7720(2004); and Yordanov, et al., J. Mater. Chem., 13(7), 1523-1525 (2003)), albumin (see Dafni, et al., Cancer Res., 62(22), 6731-6739 (2002); and Dafni, et al., NMR Biomed., 15(2), 120-131 (2002)) and cross-linked iron oxide (CLIO) (see Bulte and Kraitchman, NMR Biomed., 17(7), 484-499 (2004); and Kircher, et al., Cancer Res., 63(20), 6838-6846 (2003)). Various problems with these macromolecular MRI contrast agents are associated with their biocompatibility and/or biodistribution characteristics. Since ELP is a protein-based macromolecule. MRI agents based on ELP should have improved biocompatibility. The biodistribution of ELPs are well studied, and they demonstrate minimal uptake by the reticulo-endothelial system (RES) (e.g. liver and spleen). See Gabizon, et al., Clin. Pharmacokinet., 42(5), 419-436 (2003); and Kawai, et al., Cell Tissue Res., 292(2), 395-410 (1998). Since ELP is genetically encoded, positions for metal ion attachment, targeting elements, and enzymatically recognizable sequences can be incorporated at specific sites and with specified frequency along the ELP backbone.

Thus, the presently disclosed subject matter provides high MW MRI contrast agents by chelating one or more paramagnetic metal ions to an ELP. In some embodiments, specific sequences can be incorporated within the ELP portion of the ELP MRI contrast agent to facilitate its degradation and subsequent clearance. Other sequences that bind to or are enzymatically cross-linked into specific tissues can also be incorporated into the ELP in order to facilitate diagnosis with the ELP MRI contrast agent. Since ELP is thermally responsive, the ELP MRI contrast agent can be used with MRI for noninvasive thermometry. Further, in view of recent interest in the ability to image drug delivery (see Vigilant', et al., Magn. Reson. Med., 56(5), 1011-1018 (2006) and Viglianti, et al., Magn. Reson. Med., 51(6), 1153-1162 (2004)), and as ELP can also be used as a drug carrier, in some embodiments, the ELP MRI contrast agent can comprise a therapeutic agent or can be used in conjunction with an additional drug carrier ELP. Thus, in some embodiments, the ELP MRI contrast agent will provide an opportunity to combine imaging with drug delivery in one molecule or through the use of a mixture of similar molecules. In some embodiments. MRI and optical imaging can be accomplished by bonding paramagnetic and optical imaging moieties to an ELP to aid in surgical resection. Lastly, all the advantages of a high MW contrast agent such as blood volume determination, magnetic resonance angiography (MRA), and vascular transport determination can be taken advantage of with a biocompatible ELP MRI contrast agent.

In some embodiments, the ELP is chelated to one or more paramagnetic metal ions. In some embodiments, the paramagnetic ion is selected from the group consisting of a transition element, a lanthanide element, and an actinide element. In some embodiments, the paramagnetic metal ion is selected from the group consisting of Gd(III), Mn(II), Cu(II), Cr(III), Fe(II), Fe(III), Co(II), Er(II) Ni(II), Eu(III) and Dy(III). In some embodiments, the paramagnetic metal ion is Gd(III).

In some embodiments, the paramagnetic metal ion is chelated to the ELP via one or more amines present in the ELP amino acid sequence. In some embodiments, the amine will be the amine at the N-terminus of the ELP. In some embodiments, a Lys residue will be used as the guest resiude “Xaa” in the pentapeptide Val-Pro-Gly-Xaa-Gly (SEQ ID NO: 1) in one or more of the pentapeptide repeat sequences of the ELP. In some embodiments, the chelation will further involve the bonding of one or more residues of the ELP with a bifunctional chelator moiety. In some embodiments, one or more Lys residues in the ELP will be bonded to one or more bifunctional chelator moieties, wherein the bifunctional chelator moiety comprises one metal chelation group and one group that can interact (i.e., conjugate or bond) to the ELP peptide. In some embodiments, the group that interacts with the ELP peptide forms a covalent bond with an atom on the ELP peptide.

In particular, the bifunctional chelator moiety can have a structure of the formula:

R₁-L₁-Che

wherein:

R₁ is a reactive group;

L₁ is a linker group; and

Che is a chelator.

Suitable R₁ groups include isothiocyantato (ITC, i.e., a —N═C═S group) and active esters (i.e., —C(═O)OR₂) that can react with the primary amine of a lysine residue to form an amide linkage. For example, R₂ can comprise aryl or substituted aryl (e.g., pentafluorophenyl) or succinimide. In some embodiments. R₁ comprises an N-hydroxysuccinimide group (NHS):

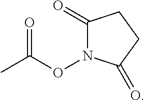

The linking group L₁ can comprise any suitable bivalent linking group. In some embodiments, L₁ is alkylene, arylene (e.g., phenylene) or a combination thereof (e.g., -aryl-alkyl- or -alkyl-aryl-alkyl- or -aryl-alkyl-aryl-). In some embodiments. L₁ is a peptide. In some embodiments, the alkylene or arylene can comprise one or more alkyl or aryl group substitutents. In some embodiments, the alkylene or arylene can comprise one or more heteroatoms. For instance, the alkylene group can comprise an ethylene glycol-based oligomer.

In some embodiments, the linker group or the reactive group comprises a degradable linkage (e.g., a peptide-based group or a disulfide linkage) to promote clearance of the paramagnetic metal ion from the body.

A number of suitable metal chelator groups are known in the art and can be used in the bifuntional chelator. In some embodiments, the chelator is diethylenetriaminepentaacetate (DTPA), the structure of which is shown below, forms a stable complex, i.e., chelates, with metal ions, e.g., the rare-earth element gadolinium (Gd³⁺), and thus acts to detoxify the metal ions.

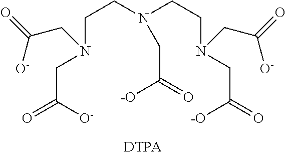

The stability constant (K) (also referred to as the “formation constant”) for Gd(DTPA)⁻² is very high (log K=22.4). The higher the log K, the more stable the complex. This thermodynamic parameter indicates that the fraction of Gd⁺³ ions that are in the unbound state will be quite small.

The molecule 1,4,7,10-tetraazacyclododecane′-N,N′,N″,N′″-tetracetic acid (DOTA) and derivatives thereof have been used to chelate metal ions. See U.S. Pat. Nos. 5,155,215; 5,087,440; 5,219,553; 5,188,816; 4,885,363; 5,358,704; 5,262,532; and Meyer et al., Invest. Radiol., 25, S53 (1990). The Gd-DOTA complex has been thoroughly studied in laboratory tests involving animals and humans. The complex is conformationally rigid, has an extremely high formation constant (log K=28.5), and at physiological pH possess very slow dissociation kinetics.

In addition to DTPA and DOTA, a number of other metal chelators can be used in the presently disclosed ELP contrast agents. See, for example, PCT International Patent Publication No. WO96/23526, which is herein incorporated by reference in its entirety. Thus, suitable chelator groups also include, but are not limited to, 1,2,7,10-tetraazacyclododecane-1,4,7-triacetic acid (DO3A), trans-1,2-cyclohexanediamine tetraacetic acid (CDTA), ethylenediaminetetraacetic acid (EDTA), and tris-(2-aminoethyl)amine (TETA).

In some embodiments, the ELP can comprise a peptide sequence or sequences in addition to the polymer of pentapeptide Val-Pro-Gly-Xaa-Gly (SEQ ID NO: 1). In some embodiments, the ELP portion of the MRI contrast enhancement agent can comprise an enzymatically recognized reaction site. In some embodiments, the enzymatically recognized site is a degradation site. Since the ELP is genetically encoded, enzymatic degradation sites can be easily incorporated into the ELP's sequence. The enzyme can be present in circulation or in specific tissues. The degradation of the ELP can increase its clearance rate and improve its safety profile. The sequence can be incorporated throughout the ELP's backbone or at specific sites such as between the blocks of an ELP_{BC}. The degradation of an ELP MRI agent in specific tissues or at disease sites can also facilitate a decrease in signal that could be used for diagnosis.

In some embodiments, the enzymatically recognized reaction site is a cross-linking site. For example, in some embodiments, a peptide sequence can be present in the ELP peptide that is recongnized by transglutaminase (TG). See Mazooz, et al., Cancer Res., 65(4), 1369-1375 (2005). When exposed to the enzyme, the ELP is crosslinked into the tissue expressing the enzyme and is retained longer than if no crosslink was formed. The higher concentration of ELP-Gd conjugate can then be detected with MRI. The crosslinking enzymes can be upregulated in disease states such as cancer or wound healing and therefore facilitate in diagnosis of the disease with the ELP contrast enhancement agent.

Specific enzymes can link two or more ELP molecules together through an enzymatically recognized reaction site, thereby increasing the ELP's MW. The higher MW ELP MRI can have a slower rotational correlation time and therefore an increased relaxivity, which potentially can generate a detectable signal change. Enzymes that crosslink ELPs to one another can be upregulated in disease states such as tumors or wound healing and therefore facilitate in diagnosis with ELP contrast agents.

In some embodiments, a targeting group can be incorporated into the ELP MRI contrast agent. Again, since the ELP is genetically encoded, affinity targeted elements such as single chain antibody fragments or peptides (e.g. RGD, NGR) can be incorporated into the ELP sequence to target specific tissues or disease sites. These targeting elements can be presented in single or multiple copies on an ELP or in the corona of a micelle formed with ELP_{BC}s.

In some embodiments, the ELP MRI contrast agent can form a micelle structure. For example, the micelle structure can have a larger effective diameter (about 60 nm) than a single molecule (about 10 nm). Thus, the micelle moiety can have a longer plasma half-life than the single molecule, which can be beneficial in certain diagnostic procedures, such as blood volume determination. In some embodiments, the micelle agent will comprise ELP_{BC}s, wherein the ELP_{BC} can self-assemble due to its inherent amphiphilic nature. Multiple lysine residues can be used in the high Tₜ (i.e., the solvated block) of the ELP_{BC} to bond an imaging agent (paramagnetic or optical).

In some embodiments, the ELP agent can comprise a radioactive isotope (i.e., a radionuclide) so that the ELP agent can be used as an agent for positron emission tomography (PET) or single photon emission computed tomography (SPECT). The ELP agent can be used purely as a PET or SPECT agent or can be used as a PET or SPECT agent in addition to being used as an MRI imaging agent. As will be understood by one of skill in the art, a number of radionuclides can be used as the isotope for a PET or SPECT agent. Suitable radionuclides include, but are not limited to, ¹⁸F, ⁶⁴Cu, ¹²⁴I, ¹¹¹In, ⁶⁷Ga, ²¹²Bi, ²⁰¹Tl and ^{99m}Tc. In some embodiments, the ELP PET agent comprises an isotope selected from ¹⁸F, ⁶⁴Cu, and ¹²⁴I. In some embodiments, the ELP SPECT agent comprises ^{99m}Tc. The ELP PET or SPECT agent can also comprise a therapeutic agent, a targeting agent, or an optical imaging agent.

### III. Methods of Using ELP MRI Contrast Agents

In some embodiments, the presently disclosed subject matter provides a method of imaging a biological sample, the method comprising contacting the biological sample with a contrast enhancement agent, wherein the contrast enhancement agent comprises an elastin-like polypeptide (ELP) and one or more paramagnetic metal ions; and rendering a magnetic resonance image of the sample. In some embodiments, the biological sample is one of a cell, a tissue, an organ, and a subject (e.g., a patient, such as a human patient).

In some embodiments, the method of generating a visible image of the biological sample further indicates the presence of a disease state. In some embodiments, the disease state is cancer or atherosclerosis. Thus, in some embodiments the biological sample comprises a tumor or neoplasm. Representative neoplasms that can be targeted by the instant methods are selected from the group consisting of benign intracranial melanomas, arteriovenous malformation, angioma, macular degeneration, melanoma, adenocarcinoma, malignant glioma, prostatic carcinoma, kidney carcinoma, bladder carcinoma, pancreatic carcinoma, thyroid carcinoma, lung carcinoma, colon carcinoma, rectal carcinoma, brain carcinoma, liver carcinoma, breast carcinoma, ovary carcinoma, solid tumors, solid tumor metastases, angiofibromas, retrolental fibroplasia, hemangiomas. Karposi's sarcoma, and combinations thereof.

In some embodiments, the ELP can be labeled (i.e., conjugated) with both a paramagnetic metal ion and with one or more additional optical imaging agents, such as, but not limited to, a fluorescent dye. In some embodiments, the ELP can also be targeted to a specific site (e.g., tissue, cell or disease state) in vivo. Both targeted and enzyme crosslinked ELP versions can be retained longer than simple ELPs in the tumor. This retention can be to specific interaction with a tumor antigen (e.g., αᵥβ₃, APN, or folate receptor) and/or enzymatic incorporation into the tumor (the enzyme will be specific to the tumor such as TG). MRI can then be used to identify the tumor location and to plan surgical resection. During surgical resection, the optical probe (e.g., NIR probe or fluorescein) can aid in identifying the border between tumor and normal tissue and therefore improve the resection of a tumor and limit residual disease left in the tumor margin. Tumors often recur in the margin, ultimately leading to treatment failure. Therefore, the ability to identify tumor from normal tissue during surgical resection is widely applicable.

In some embodiments, the disease state relates to heart disease or other disease states caused by blockages (e.g., arterial plaques) in the circulatory pathway. In some embodiments, the method of generating a visible image indicates the progress or lack thereof of a process related to wound healing.

In some embodiments, the ELP can function as a macromolecular drug carrier. See Meyer, et al., Cancer Res., 61(4), 1548-1554 (2001); Dreher, et al., J. Control. Release, 91(1-2), 31-43 (2003); Chilkoti, et al., Adv. Drug Deliv. Rev., 54(8), 1093-1111 (2002); and Chilkoti, et al., Adv. Drug Deliv. Rev., 54(5), 613-630 (2002). Thus, in some embodiments, imaging and drug delivery functions can be combined to image the distribution of drugs to specific tissues in the body.

In some embodiments of the instant method, a therapeutic agent comprises an ELP conjugated to a therapeutic composition (also referred to herein as an “active agent”) in addition to being conjugated to one or more paramagnetic metal ions. As used herein, the term “therapeutic composition” refers to a polypeptide (referred to herein as a “therapeutic polypeptide”) or other molecule than when introduced into a target results in a therapeutically beneficial effect. Representative therapeutic compositions comprise chemotherapeutic agents, toxins, radiotherapeutics, and combinations thereof. Each agent is loaded in a total amount effective to accomplish the desired result in the target, whether the desired result be imaging the target or treating the target.

Chemotherapeutics useful as active agents are typically small chemical entities produced by chemical synthesis. Chemotherapeutics include cytotoxic and cytostatic drugs. Chemotherapeutics can include those that have other effects on cells including, but not limited to reversal of a transformed state to a differentiated state or those that inhibit cell replication. Exemplary chemotherapeutic agents include, but are not limited to anti-tumor drugs, cytokines, anti-metabolites, alkylating agents, hormones, and the like.

Additional examples of chemotherapeutics include common cytotoxic or cytostatic drugs such as, for example, methotrexate (amethopterin), doxorubicin (adrimycin), daunorubicin, paclitaxel, cytosine arabinoside, etoposide, 4-fluorouracil, 5-fluorouracil, melphalan, chlorambucil, and other nitrogen mustards (e.g. cyclophosphamide), cis-platinum, vindesine (and other vinca alkaloids), mitomycin and bleomycin. Other chemotherapeutics include, but are not limited to purothionin (barley flour oligopeptide), macromomycin, 1,4-benzoquinone derivatives, trenimon, steroids, aminopterin, anthracyclines, demecolcine, etoposide, mithramycin, daunomycin, vinblastine, neocarzinostatin, macromycin, α-amanitin, and the like. The use of combinations of chemotherapeutic agents is also provided in accordance with the presently disclosed subject matter. In some embodiments, the chemotherapeutic agent is selected from the group consisting of an anti-tumor drug, a cytokine, an anti-metabolite, an alkylating agent, a hormone, methotrexate, doxorubicin, daunorubicin, cytosine arabinoside, etoposide, 4-fluorouracil, 5-fluorouracil, melphalan, chlorambucil, a nitrogen mustard, cyclophosphamide, cis-platinum, vindesine, vinca alkaloids, mitomycin, bleomycin, purothionin, macromomycin, 1,4-benzoquinone derivatives, trenimon, steroids, aminopterin, anthracyclines, demecolcine, etoposide, mithramycin, doxorubicin, daunomycin, vinblastine, neocarzinostatin, macromycin, α-amanitin, and combinations thereof.

Toxins can also be employed as active agents. Once delivered, the toxin moiety can kill cells in the target. Toxins are generally complex toxic products of various organisms including bacteria, plants, etc.

Exemplary toxins include, but are not limited to coagulants such as Russell's Viper Venom, activated Factor IX, activated Factor X, and thrombin; and cell surface lytic agents such as phospholipase C and cobra venom factor (CVF), which should lyse neoplastic cells directly. Additional examples of toxins include, but are not limited to ricin, ricin A chain (ricin toxin), Pseudomonas exotoxin (PE), diphtheria toxin (DT), bovine pancreatic ribonuclease (BPR), pokeweed antiviral protein (PAP), abrin, abrin A chain (abrin toxin), gelonin (GEL), saporin (SAP), modeccin, viscumin, and volkensin. In some embodiments, the toxin is selected from the group consisting of Russell's Viper Venom, activated Factor IX, activated Factor X, thrombin, phospholipase C. cobra venom factor, ricin, ricin A chain, Pseudomonas exotoxin, diphtheria toxin, bovine pancreatic ribonuclease, pokeweed antiviral protein, abrin, abrin A chain, gelonin, saporin, modeccin, viscumin, volkensin, and combinations thereof.

Radiotherapeutic agents can also be employed as active agents. Exemplary radiotherapeutic agents include, but are not limited to ⁴⁷Sc, ⁶⁷Cu, ⁹⁰Y, ¹⁰⁹Pd, ¹²³I, ¹²⁵I, ¹³¹I, ¹¹¹In, ¹⁸⁶Re, ¹⁹⁹Au, ²¹¹At, ²¹²Pd, and ²¹²Bi. Other radiotherapeutic agents that can be employed include ³²P and ³³P, ⁷¹Ge, ⁷⁷As, ¹⁰³Pb, ¹⁰⁵Rh, ¹¹¹Ag, ¹¹⁹Sb, ¹²¹Sn, ¹³¹Cs, ¹⁴³pr, ¹⁶¹Tb, ¹⁷⁷Lu, ¹⁹¹Os, ^{193M}Pt, ¹⁹⁷Hg, all beta negative and/or auger emitters. Other representative radiotherapeutic agents include ⁹⁰Y, ¹³¹I, ²¹¹At, and ²¹²Pb/²¹²Bi. In some embodiments, the radiotherapeutic agent is selected from the group consisting of ⁴⁷Sc, ⁶⁷Cu, ⁹⁰Y, ¹⁰⁹Pd, ¹²³I, ¹²⁵I, ¹³¹I, ¹⁸⁶Re, ¹⁸⁸Re, ¹⁹⁹Au, ²¹¹At, ²¹²Pb, ²¹²Bi, ³²P, ³³P, ⁷¹Ge, ⁷⁷As, ¹⁰³Pb, ¹⁰⁵Rh, ¹¹¹Ag, ¹¹⁹Sb, ¹³¹Sn, ¹³¹Cs, ¹⁴³Pr, ¹⁶¹Tb, ¹⁷⁷Lu, ¹⁹¹Os, ^{193M}Pt, and ¹⁹⁷Hg.

In some embodiments, the method further comprises exposing the biological sample (for example, a tumor or neoplasm) to a therapeutic dose of ionizing radiation. As used herein, the term “ionizing radiation” is meant to refer to any radiation where an electron. X-ray, gamma ray, or nuclear particle has sufficient energy to remove an electron or other particle from an atom or molecule, thus producing an ion and a free electron or other particle. Examples of such ionizing radiation include, but are not limited to gamma rays. X-rays, protons, electrons, and alpha particles. Ionizing radiation is commonly used in medical radiotherapy and the specific techniques for such treatment will be apparent to a skilled practitioner in the art. Dosages and treatment regimens for radiotherapy are also known to those of skill in the art.

In some embodiments, the method involves the targeted delivery of the ELP MRI contrast agent to a particular cell, tissue, or organ (e.g., a cell, tissue or organ) that expresses a particular recognition feature, enzyme, or that is indicative of a particular disease state.

In some embodiments, the ELP MRI contrast agents can also be used as “blood pool” agents in blood volume determination, in magnetic resonance angiography (MRA), and in determining vascular transport (e.g., K^{trans}) and extravascular-extracellular volume fraction (vₑ) in dynamic contrast enhanced magnetic resonance imaging (DCE-MRI).

### IV. Subjects

The methods and compositions disclosed herein can be used on a target either in vitro (for example, on isolated cells or tissues) or in vivo in a subject (i.e. living organism, such as a patient). In some embodiments, the subject is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As such, the methods of the presently disclosed subject matter are particularly useful in the treatment of warm-blooded vertebrates. Thus, the presently disclosed subject matter concerns mammals and birds. More particularly provided is the treatment of mammals such as humans, as well as those mammals of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos or as pets, as well as fowl, and more particularly domesticated fowl, for example, poultry, such as turkeys, chickens, ducks, geese, guinea fowl, and the like, as they are also of economic importance to humans. Thus, also contemplated is the treatment of livestock including, but not limited to domesticated swine (pigs and hogs), ruminants, horses, poultry, and the like.

### V. Formulation

The compositions of the presently disclosed subject matter comprise in some embodiments a composition that includes a pharmaceutically acceptable carrier. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the subject; and aqueous and non-aqueous sterile suspensions that can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are sodium dodecyl sulfate (SDS), in one example in the range of 0.1 to 10 mg/ml, in another example about 2.0 mg/ml; and/or mannitol or another sugar, for example in the range of 10 to 100 mg/ml, in another example about 30 mg/ml; and/or phosphate-buffered saline (PBS).

It should be understood that in addition to the ingredients particularly mentioned above, the formulations of this presently disclosed subject matter can include other agents conventional in the art having regard to the type of formulation in question. For example, sterile pyrogen-free aqueous and non-aqueous solutions can be used.

### VI. Administration

Suitable methods for administration of a composition of the presently disclosed subject matter include, but are not limited to intravenous and intratumoral injection. Alternatively, a composition can be deposited at a site in need of treatment in any other manner, for example by spraying a composition comprising a composition within the pulmonary pathways. The particular mode of administering a composition of the presently disclosed subject matter depends on various factors, including the distribution and abundance of cells to be imaged and/or treated and mechanisms for metabolism or removal of the composition from its site of administration. For example, relatively superficial tumors can be injected intratumorally. By contrast, internal tumors can be imaged and/or treated following intravenous injection.

In one embodiment, the method of administration encompasses features for regionalized delivery or accumulation at the site to be imaged and/or treated. In some embodiments, a composition is delivered intratumorally. In some embodiments, selective delivery of a composition to a target is accomplished by intravenous injection of the composition followed by hyperthermia treatment of the target.

For delivery of compositions to pulmonary pathways, compositions of the presently disclosed subject matter can be formulated as an aerosol or coarse spray. Methods for preparation and administration of aerosol or spray formulations can be found, for example, in U.S. Pat. Nos. 5,858,784; 6,013,638; 6,022,737; and 6,136,295.

### VII. Doses

An effective dose of a composition of the presently disclosed subject matter is administered to a subject. An “effective amount” is an amount of the composition sufficient to produce adequate imaging and/or treatment. Actual dosage levels of constituents of the compositions of the presently disclosed subject matter can be varied so as to administer an amount of the composition that is effective to achieve the desired effect for a particular subject and/or target. The selected dosage level will depend upon the activity of the composition and the route of administration.

After review of the disclosure herein of the presently disclosed subject matter, one of ordinary skill in the art can tailor the dosages to an individual subject, taking into account the particular formulation, method of administration to be used with the composition, and nature of the target to be imaged and/or treated. Such adjustments or variations, as well as evaluation of when and how to make such adjustments or variations, are well known to those of ordinary skill in the art.

### EXAMPLES

The following Examples have been included to illustrate modes of the presently disclosed subject matter. Certain aspects of the following Examples are described in terms of techniques and procedures found or contemplated by the present co-inventors to work well in the practice of the presently disclosed subject matter. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the presently disclosed subject matter.

### Materials and Methods

ELP peptides can be prepared and purified using a variety of methods known in the art. ELP peptides can be prepared using organic synthetic methodology (e.g., solid phase peptide synthesis). Alternatively, the protein-based polymers can be prepared via a biosynthetic approach using current recombinant DNA methodologies. Using this approach, a gene encoding the desired peptide sequence is constructed, artificially inserted into, and then translated in a host organism. The host can be eukaryotic (e.g., yeast), plant, or prokaryotic (e.g., bacteria). Usually, the host will be microbial, where the resulting protein can then be purified, often in large amounts, from cultures grown in fermentation reactors. Recombinant DNA can be used to create synthetic genes encoding multiple repeating units of a given peptide sequence and these synthetic genes may themselves be polymerized to create even longer coding sequences, resulting in protein-based polymers of greater length. See, for example, U.S. Pat. No. 5,854,387. Methods of preparing and isolating fusion proteins comprising ELP using recombinant expression systems has also been previously described. See U.S. Pat. No. 6,852,834. Methods of preparing therapeutic agent conjugates of ELPs are described in U.S. Pat. No. 6,582,926. Preparation and use of ELPs as non-invasive thermometry agents is described in PCT International Publication No. WO2006/001806.

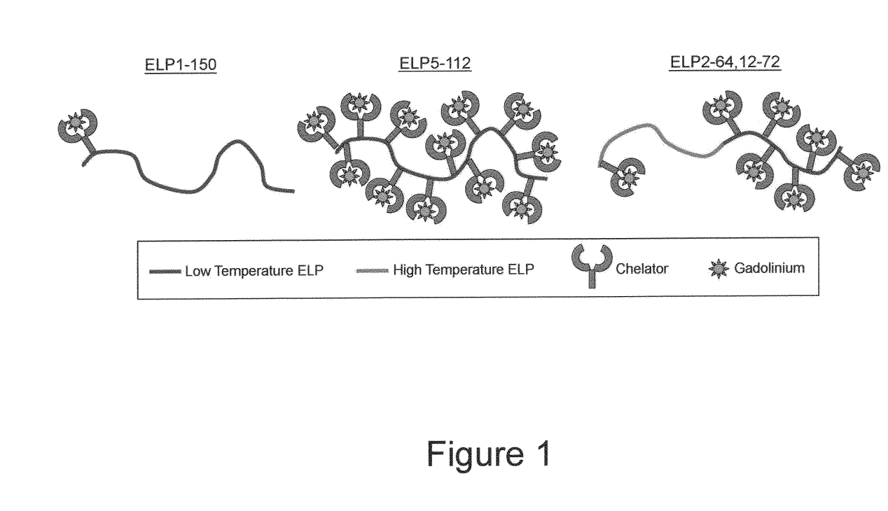

Three different ELP peptides were prepared for use in synthesizing and studying ELP MRI contrast enhancement agents. All ELPs have an amine group at their N-terminus. In addition to this amine group, lysine residues were placed at specific sites along the ELP backbone. In the simplest design, one lysine residue was positioned near the N-terminus of ELP1-150 (SEQ ID NO: 2). ELP5-112 (SEQ ID NO: 3) has 17 lysines placed throughout the ELP sequence. As described hereinabove. ELP_{BC}s have been shown to self-assemble into spherical micelles (diameter ˜60 nm) when the solution temperature is between the Tₜ of both blocks. ELP2-64,12-72 (SEQ ID NO: 4) is a ELP_{BC}, which has 8 lysine residues within the C-terminal block and one lysine at the N-terminus. A schematic drawing depicting the MRI contast agents prepared from SEQ ID NOS: 2-4 is shown in FIG. 1.

### Example 1

### Preparation of ELP-Gd Conjugates

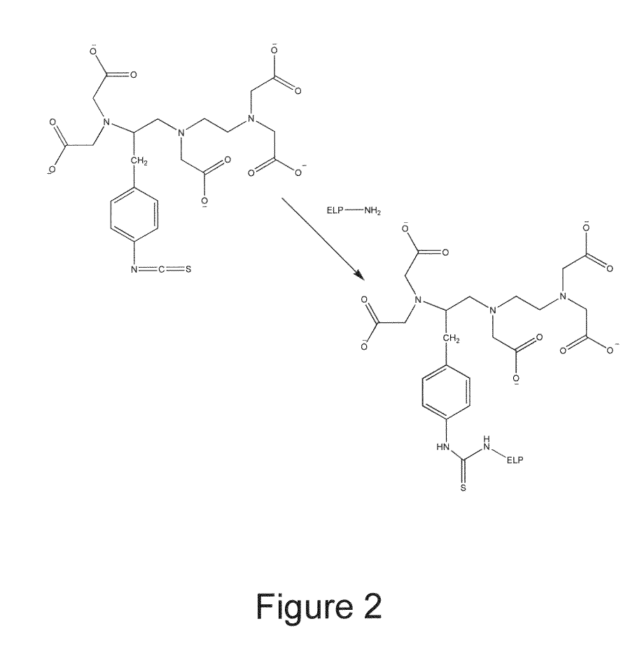

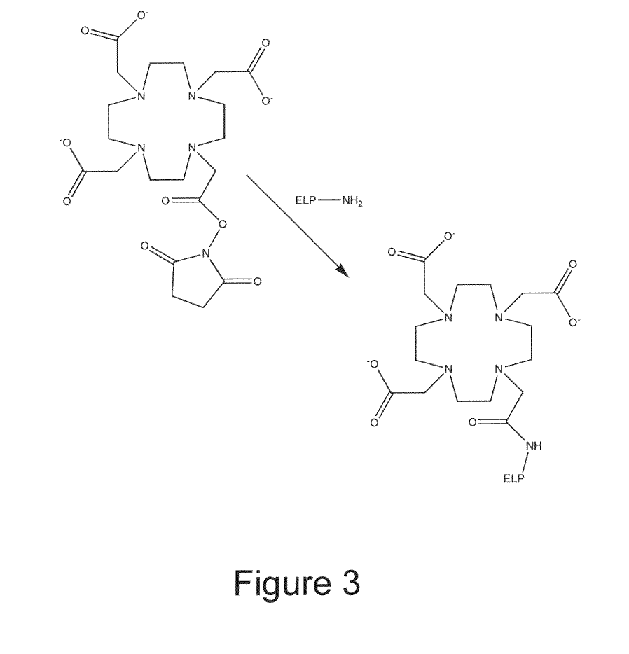

Conjugation of ELP to bifunctional chelator: A scheme showing the conjugation of DTPA-ITC to a generic ELP is shown in FIG. 2, while a scheme showing the conjugation of DOTA-NHS to an ELP is shown in FIG. 3.

More particularly, the conjugation of DTPA-ITC to ELP1-150 (SEQ ID NO: 2) was carried out by preparing a 150 μM solution of ELP1-150 (SEQ ID NO: 2) in 100 mM sodium bicarbonate buffer (pH=8.4). An aqueous solution comprising a five-fold molar excess of DTPA-ITC (Macrocyclics, Dallas, Tex., United States of America) was added and the conjugation reaction was allowed to proceed for 2 hours at room temperature.

Similarly, the conjugation of DOTA-NHS to ELP1-150 (SEQ ID NO: 2) was carried out by preparing a 150 μM solution of ELP1-150 (SEQ ID NO: 2) in 100 mM sodium bicarbonate buffer (pH=8.4). A DMSO solution containing a five-fold molar excess of DOTA-NHS (Macrocyclics, Dallas, Tex., United States of America) was added. The conjugation was allowed to proceed for 2 hours at room temperature.

Purification of ELP chelator conjugates and chelation of Gd: Each conjugation reaction mixture was purified to separate any remaining free chelator from the ELP-chelator conjuguates and any free ELP by inverse transition cycling. Sodium chloride (NaCl) was added to the reaction mixture (final concentration=1.33 to 3 M) to aggregate the ELP by depressing its Tₜ below the solution temperature (the Tₜ of the ELP is dependent upon co-solutes). The resulting solution was centrifuged (16,100×g) at or above room temperature for 10 min. The supernatant was discarded and the pellet was resuspended in 100 mM sodium acetate buffer (pH=5.0) containing a 2-fold molar excess of Gd per lysine residue. The solution was stirred overnight.

Purification of ELP-Gd: The ELP-Gd conjugate and free ELP were separated from free Gd by inverse transition cycling by adding NaCl (final concentration=1.33 to 3 M) to aggregate the ELP by depressing its Tₜ below the solution temperature. The resulting solution was centrifuged (16,100×g) at or above room temperature for 10 min. The supernatant was discarded and the pellet was resuspended in cold PBS and centrifuged (16,100×g) at 4° C. for 5 minutes to remove any insoluble matter. The resulting supernatant was then further purified by size exclusion chromatography with a PD-10 column (Amersham Biosciences, Piscataway, N.J., United States of America) to ensure that all the free Gd was removed. The purified conjugate was then concentrated by inverse transition cycling and stored at −20° C. in phosphate buffered saline (PBS) at a concentration of about 500 μM, until further use.

### Example 2

### Characterization of ELP-GD

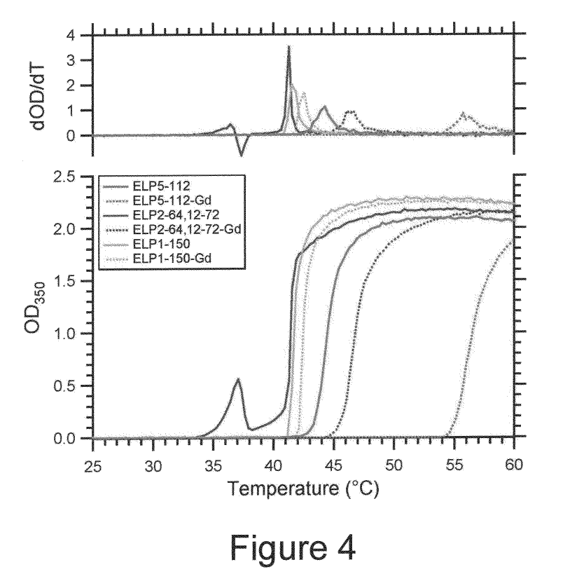

The ELPs' thermal properties were characterized by monitoring the absorbance of an ELP solution using temperature dependent UV-Vis spectrophotometry. The turbidity profile (i.e., optical density (OD) versus temperature) for the ELP-Gd conjugates and their parent ELPs is shown in FIG. 4.

The ELP solution was transparent at low temperatures, but as the temperature was increased, the ELP underwent its inverse temperature phase transition and formed large aggregates that increased the absorbance of the solution (i.e., optical density). The Tₜ is defined as the temperature at the maximum in dOD/dT for the bulk aggregation. Overall, attaching Gd to an ELP increases its Tₜ. The ELP_{BC} self-assembly was affected by the attachment of Gd. Without being bound to any one particular theory, the effects on ELP_{BC} self-assembly can be due to the influence of the Gd ion and the chelator on the hydrophilicity of the low Tₜ block.

The thermal properties of the ELPs and the efficiency of the Gd conjugation reactions are summarized in Table 1. The molar ratio of Gd to ELP was determined with inductively coupled plasma atomic emission spectrophotometry (ICPAES). The degree of labeling (DOL) was determined by dividing Gd/ELP by ratio of amine/ELP.

Attaching the bifunctional chelator to the ELP in the first step of the conjugation procedure results in a marked increase in the ELP's Tₜ (data not shown). Subsequent chelation of gadolinium reduces the ELP's Tₜ, but the Tₜ remains elevated above the parent EPL's Tₜ. After chelation of Gd with DOTA, Tₜ increases 0.7° C. per lysine residue. The ELPs comprising the DOTA-NHS bifunctional linker had a much higher degree of labeling (DOL) than those comprising the DTPA-ITC linker. Since the DOL was calculated from gadolinium content, it is possible that the amine labeling and/or chelation of Gd is not efficient for DTPA-ITC (DOL=0.06%). However, the DOTA-NHS conjugation strategy results in a very high DOL (between about 41% and about 98%).

TABLE 1
Summary of ELP-Gd conjugation efficiency
and thermal properties.

| | MW | Lys/ | Amine/ | chela- | Gd/ | DOL | Tₜ | Tₜ-Gd |
|---|---|---|---|---|---|---|---|---|
| ELP | (kDa) | ELP | ELP | tor | ELP | (%) | (° C.) | (° C.) |
| ELP1-150 | 59.4 | 1 | 2 | DTPA | 0.06 | 0.03 | 41.5 | 42.5 |
| (SEQ ID | | | | | | | | |
| NO: 2) | | | | | | | | |
| ELP1-150 | 59.4 | 1 | 2 | DOTA | 0.82 | 41 | 41.5 | 42.5 |
| (SEQ ID | | | | | | | | |
| NO: 2) | | | | | | | | |
| ELP2-64, | 55.1 | 9 | 10 | DOTA | 9.4 | 94 | 41.3 | 46.7 |
| 12-72 | | | | | | | | |
| (SEQ ID | | | | | | | | |
| NO: 4) | | | | | | | | |
| ELP5-112 | 47.1 | 17 | 18 | DOTA | 17.6 | 98 | 44.3 | 55.7 |
| (SEQ ID | | | | | | | | |
| NO: 3) | | | | | | | | |

### Example 3

### MRI with ELP-Gd Conjugates

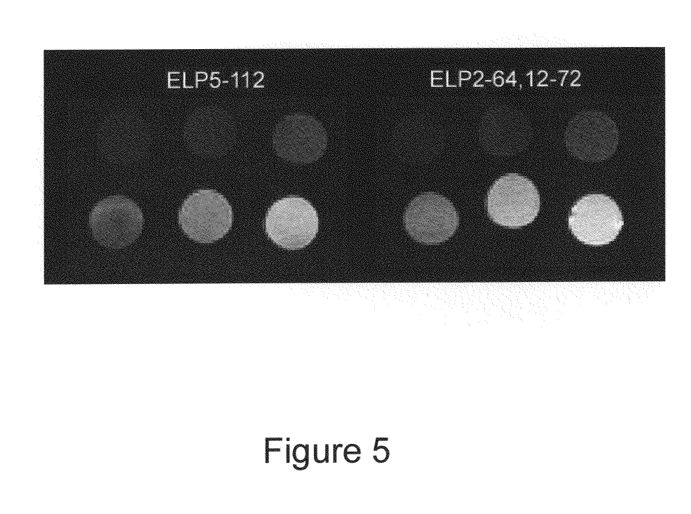

An image of two different ELP-Gd conjugates at several different concentrations is shown in FIG. 5. The image was taken with a T1-weighted spin echo sequence and a repetition time (Tr) of 150 ms. At this repetition time, a higher concentration of gadolinium created a more intense signal. However, the influence of Tr on signal intensity is complex, such that it is best to determine the longitudinal relaxation rate, T1, for each concentration of ELP-Gd conjugate.

The signal intensity for a T1-weighted image may be approximated with the following equation to gain T1.

Signal=PD[1−exp(−Tr/T1)]+background (1)

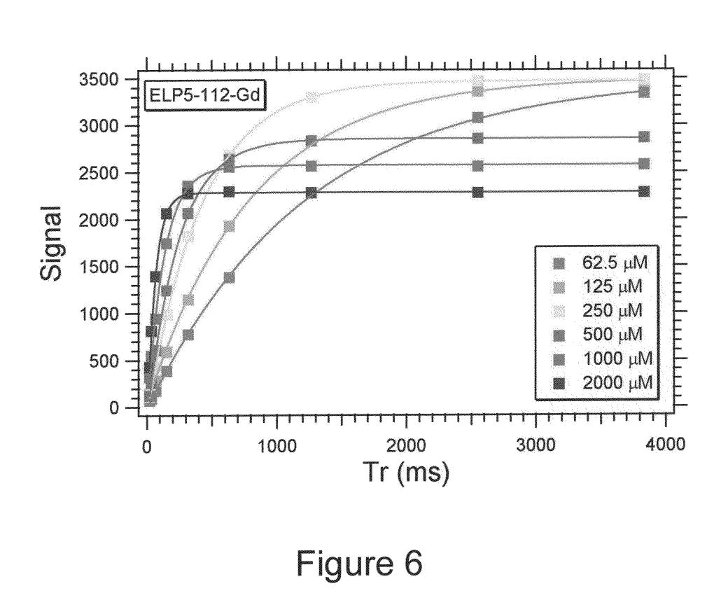

PD is the proton density, while the background is the intensity at zero gadolinium concentration. A plot of the signal versus Tr is shown in FIG. 6 for the ELP5-112 (SEQ ID NO: 3)-Gd conjugate. Samples with a higher concentration of gadolinium relax the protons faster than samples with a lower concentration of gadolinium.

The relaxivity (R) describes the ability of a contrast agent to relax protons, often generating greater signal intensity, and is defined by the following equation.

$$R = \frac{Δ ( 1 / T 1 )}{Δ C} \qquad ( 2 )$$

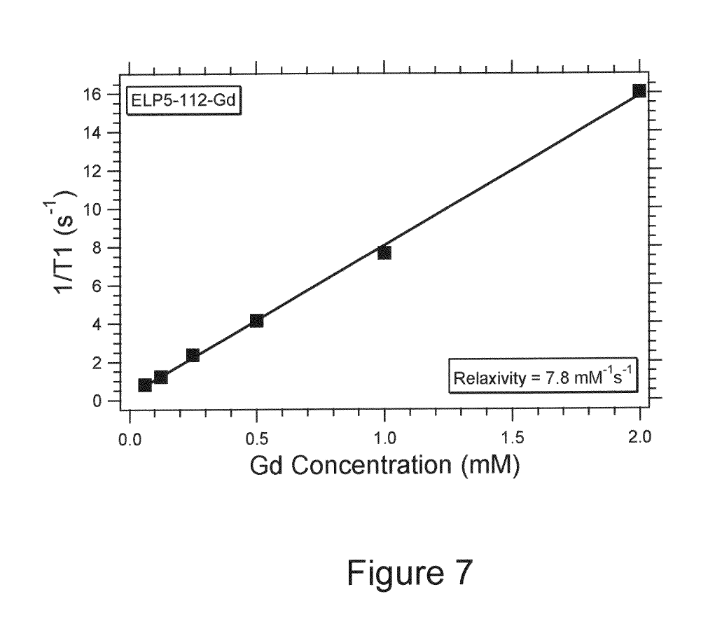

Concentration (C) is normally expressed in terms of gadolinium rather than the contrast agent to more readily compare between different contrast agents. The relationship between T1 relaxation rate and gadolinium concentration is known as the relaxivity (r1) and is shown in FIG. 7 for the ELP5-112 (SEQ ID NO: 3)-Gd conjugate.

A summary of the ELP-Gd conjugates' relaxivity is shown in Table 2. The relaxivity is expressed in terms of both gadolinium and ELP concentration. The prospective doses are approximated based on published reports of other macromolecular contrast agents and the relaxivities of the presently disclosed ELP-Gd conjugates.

TABLE 2
Summary of ELP-Gd conjugate's relaxivity (R).

| | r1 (mM⁻¹ s⁻¹) | | r1 (mM ELP⁻¹ s⁻¹) | | dose/mouse |
|---|---|---|---|---|---|
| | 2T | 7T | 2T | 7T | (mg ELP) |
| ELP1-150 | 8.4 | 4.7 | 6.8 | 3.9 | 48 to 80 |
| (SEQ ID NO: 2) | | | | | |
| ELP2-64, 12-72 | 8.1 | 4.3 | 76.1 | 40.4 | 5 to 9 |
| (SEQ ID NO: 4) | | | | | |
| ELP5-112 | 7.8 | 4.3 | 137.3 | 75.7 | 3 to 5 |
| (SEQ ID NO: 3) | | | | | |

The approximately 2-fold higher relaxivity of the presently disclosed ELP-Gd conjugates over clinically available contrast agents (−4 mM⁻¹ s⁻¹ at 1.5 T) implies that less gadolinium can be injected to generate a sufficient signal. However, gadolinium is a smaller weight percent of the present ELP agents than of the low MW contrast agents, such that the total dose of ELP agent could be high. ELP5-112 (SEQ ID NO: 3) has the highest weight percent of gadolinium and therefore has the lowest dose requirement of about 3 to 5 mg per mouse (higher doses are required for larger subjects, such as humans). Doses of 3 to 5 mg of ELP per mouse have been routinely administered to mice for 8 years without overt reactions from the mice, suggesting that ELP5-112 (SEQ ID NO: 3) can be used to prepare a viable MRI contrast enhancement agent.

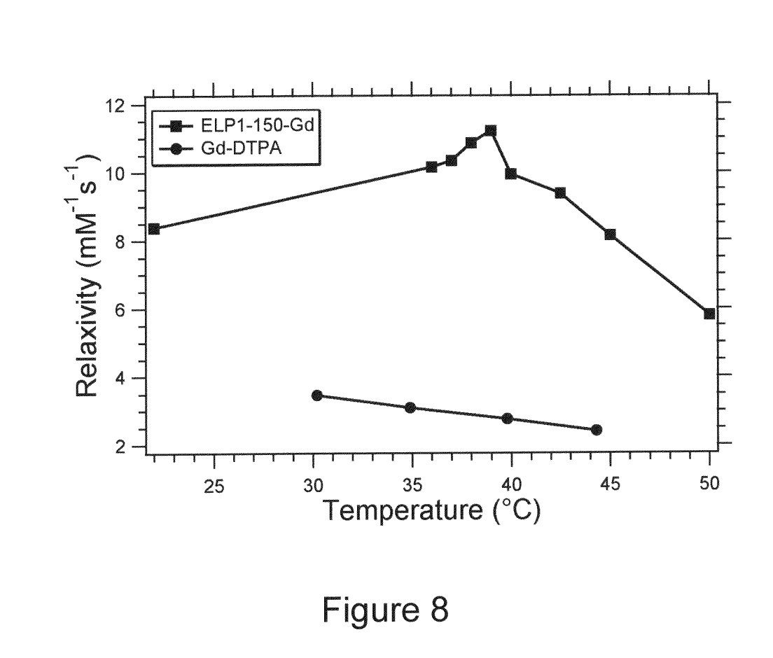

The change in relaxivity of the ELP1-150 (SEQ ID NO: 2)-Gd contrast agent was investigated over a range of temperatures to determine if the ELP can be used as a noninvasive thermometry contrast agent. The relaxivity as a function of temperature is shown in FIG. 8. The relaxivity of the ELP1-150 (SEQ ID NO: 2)-Gd conjugate increased as the solution was heated up to the ELP's phase transition, then the relaxivity decreased up to 50° C. The change in relaxivity suggests that the ELP can be used for noninvasive thermometry.

### Example 4

### MRA with ELP-Gd Conjugates

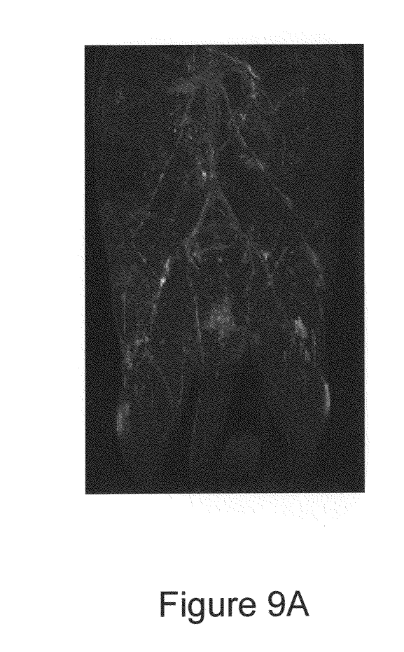

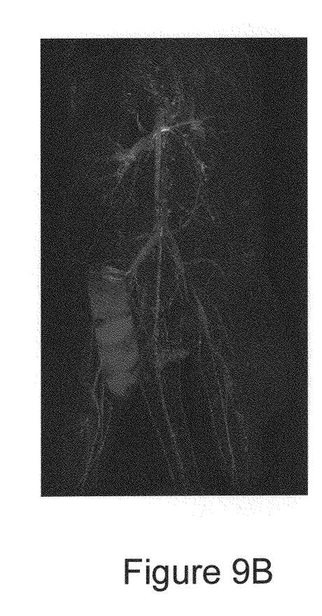

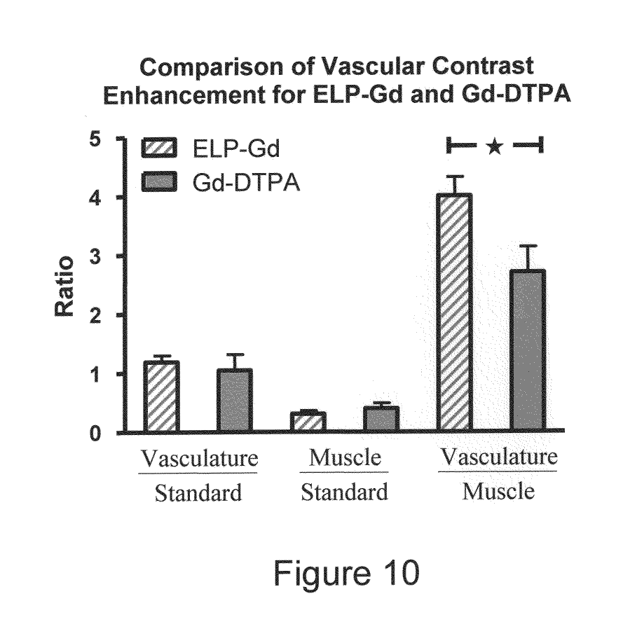

Contrast-enhanced magnetic resonance angiography (MRA) can obtain better contrast between blood vessels and surrounding tissue through the use of high MW contrast agents. To demonstrate the utility of ELP-Gd conjugates in MRA, an ELP-Gd conjugate was prepared using ELP5-112 (SEQ ID NO: 3)-DOTA. Sixteen week old BALB/c nude mice (Charles River Laboratories, Wilmington, Mass., United States of America) were injected either with Gd-DTPA (MAGNEVIST®, Bayer HealthCare Pharmaceuticals Inc., Wayne, N.J., United States of America) at a dose of 0.3 mmol Gd/kg or the ELP-Gd conjugate at a dose of 0.03 mmol Gd/kg. The mice were imaged in a Bruker 7T magnet (Bruker Corporation, Billerica, Mass., United States of America) under isofluorane anesthesia using a FLASH 3D sequence. FIG. 9A shows the MRI image of a Gd-DTPA injected mouse. An MRI standard can be seen in the bottom right corner between the tail and the right leg. FIG. 9B shows the MRI image of an ELP-Gd injected mouse. The MRI standard is over the left leg of the mouse in FIG. 9B. FIG. 10 is a graph of the ratios of various signal intensities for the different mice. As indicated by the graph, a significant improvement in vascular contrast can be observed using ELP-Gd. The signal intensity also persists over a longer period of time, allowing for improved resolution with longer scan times.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A contrast enhancement agent comprising an elastin-like polypeptide (ELP) and one or more paramagnetic metal ion.

2. The contrast enhancement agent of claim 1, wherein the paramagnetic metal ion is selected from the group consisting of a transition element, a lanthanide element, and an actinide element.

3. The contrast enhancement agent of claim 1, wherein the paramagnetic metal ion is selected from the group consisting of Gd(III), Mn(II), Cu(II), Cr(III), Fe(II), Fe(III), Co(II), Er(II), Ni(II), Eu(III) and Dy(III).

4. The contrast enhancement agent of claim 3, wherein the paramagnetic metal ion is Gd(III).

5. The contrast enhancement agent of claim 1, further comprising one or more bifunctional chelators.

6. The contrast enhancement agent of claim 5, wherein the one or more bifunctional chelators each comprise a chelator selected from the group consisting of diethylenetriaminepentaacetate (DTPA), 1,4,7,10-tetraazacyclododecane-1,4,7,10-tetraacetic acid (DOTA), 1,2,7,10-tetraazacyclododecane-1,4,7-triacetic acid (DO3A), trans-1,2-cyclohexanediamine tetraacetic acid (CDTA), ethylenediaminetetraacetic acid (EDTA), and tris-(2-aminoethyl)amine (TETA).

7. The contrast enhancement agent of claim 6, wherein each chelator is DOTA.

8. The contrast enhancement agent of claim 5, wherein the one or more bifunctional chelators are each bonded to the ELP via a covalent linkage.

9. The contrast enhancement agent of claim 8, wherein each covalent linkage is independently selected from an amide and a thiourea.

10. The contrast enhancement agent of claim 5, wherein the one or more bifunctional chelators are each bonded to the ELP via an ELP amino group.

11. The contrast enhancement agent of claim 1, wherein the ELP comprises one or more lysine residues.

12. The contrast enhancement agent of claim 11, wherein the ELP comprises at least 9 lysine residues.

13. The contrast enhancement agent of claim 12, wherein the ELP comprises at least 17 lysine residues.

14. The contrast enhancement agent of claim 1, wherein the ELP has a molecular weight greater than about 10 kDa.

15. The contrast enhancement agent of claim 14, wherein the ELP has a molecular weight greater than about 40 kDa.

16. The contrast enhancement agent of claim 1, wherein the ELP comprises an amino acid sequence selected from the group consisting of SEQ ID NO: 2, SEQ ID NO: 3, and SEQ ID NO: 4.

17. The contrast enhancement agent of claim 16, wherein the ELP comprises SEQ ID NO: 3.

18. The contrast enhancement agent of claim 1, wherein the contrast enhancement agent comprises a plurality of paramagnetic metal ions.

19. The contrast enhancement agent of claim 18, further comprising at least 10 paramagnetic metal ions.

20. The contrast enhancement agent of claim 19, further comprising at least 18 paramagentic metal ions.

21. The contrast enhancement agent of claim 1, wherein the contrast agent forms a micelle.

22. The contrast enhancement agent of claim 1, wherein the contrast agent has a diameter of greater than 40 nm.

23. The contrast enhancement agent of claim 1, wherein the contrast agent has a relaxivity of 7.0 mM⁻¹ s⁻¹ or greater at 2T, based upon the concentration of metal ion.

24. The contrast enhancement agent of claim 1, further comprising one or more targeting agents.

25. The contrast enhancement agent of claim 24, wherein the targeting agent is selected from the group consisting of an antibody, an antibody fragment, a peptide, a small molecule, a peptidomimetic, and a nucleotide-derived aptamer.

26. The contrast enhancement agent of claim 25, wherein the peptide is a RGD sequence or a NGR sequence.

27. The contrast enhancement agent of claim 1, further comprising an enzymatically recognized reaction site.

28. The contrast enhancement agent of claim 27, wherein the enzymatically recognized reaction site is cross-linkable via enzymatic catalysis to one of the group consisting of another contrast enhancement agent, a cell, and a tissue.

29. The contrast enhancement agent of claim 27, wherein the enzymatically recognized reaction site is hydrolyzable via enzymatic catalysis.

30. The contrast enhancement agent of claim 1, further comprising a therapeutic agent.

31. The contrast enhancement agent of claim 30, wherein the therapeutic agent is a neoplastic agent.

32. The contrast enhancement agent of claim 1, further comprising an optical imaging moiety.

33. A formulation comprising:
a contrast enhancement agent of claim 1; and
a pharmaceutically acceptable carrier.

34. A method of generating a visible image of a biological sample, the method comprising:
contacting the biological sample with a contrast enhancement agent, the contrast enhancement agent comprising an elastin-like peptide (ELP) and one or more paramagnetic metal ions; and
rendering a magnetic resonance image of the sample.

35. The method of claim 34, wherein the contrast enhancement agent further comprises an optical imaging moiety, a therapeutic agent, or a combination thereof.

36. The method of claim 34, wherein the sample is one of a cell, a tissue, an organ and a subject.

37. The method of claim 36, wherein the subject is a human.

38. The method of claim 34, wherein generating a visible image of the biological sample further indicates the presence of a disease state.

39. The method of claim 38, wherein the disease state is cancer or atherosclerosis.

40. The method of claim 34, wherein generating a visible image of the biological sample further indicates the delivery of a therapeutic agent.

41. The method of claim 34, wherein contacting the biological sample further comprises targeting the biological sample with a targeting agent associated with the contrast enhancement agent.

42. The method of claim 34, wherein contacting the biological sample further comprises cross-linking the contrast agent to the biological sample via an enzymatically catalyzed reaction.

43. The method of claim 34, wherein the method is part of a procedure selected from the group consisting of blood volume determination, magnetic resonance angiography (MRA), and vascular transport determination.

44. A method of imaging and guiding a surgical resection of a biological sample, the method comprising:
contacting the biological sample with a contrast enhancement agent, the contrast enhancement agent comprising an elastin-like peptide (ELP), one or more paramagnetic metal ions, and an optical imaging moiety;
rendering a magnetic resonance image of the biological sample to identify the presence or location of a disease;
detecting the presence of the optical imaging moiety during a surgical resection of the biological sample, and
using the detected presence of the optical imaging moiety to guide the extent of the surgical resection of the biological sample, wherein guiding the extent of the surgical resection reduces the amount of disease-affected tissue or the likelihood of a recurrence of the disease compared to a surgical resection performed without the detection of the optical imaging agent.

45. The method of claim 44, wherein the optical imaging agent is selected from the group consisting of fluorescein, a fluorescein derivative, and an MR probe.

46. The method of claim 44, wherein the sample is one of a cell, a tissue, an organ and a subject.

47. The method of claim 46, wherein the subject is a human.

48. The method of claim 44, wherein the disease is cancer or atherosclerosis.

49. The method of claim 44, wherein the contrast enhancement agent further comprises a therapeutic agent, and wherein generating a visible image of the biological sample further indicates the delivery of a therapeutic agent.

50. The method of claim 44, wherein contacting the biological sample further comprises targeting the biological sample with a targeting agent associated with the contrast enhancement agent.

51. The method of claim 44, wherein contacting the biological sample further comprises cross-linking the contrast agent to the biological sample via an enzymatically catalyzed reaction.

52. The method of claim 44, wherein the method is part of a procedure selected from the group consisting of blood volume determination, magnetic resonance angiography (MRA), and vascular transport determination.

53. A method of preparing an elastin-like peptide (ELP) contrast enhancement agent, the method comprising:
providing an ELP, the ELP comprises at least one primary amine group;
providing a bifunctional chelator group, wherein the bifunctional chelator group comprises a group that can interact with the amine;
contacting the ELP and the bifunctional chelator group such that the bifunctional chelator group interacts with the amine to form an ELP-chelator conjugate;
providing a paramagnetic metal ion; and
contacting the ELP-chelator conjugate with the paramagnetic metal ion to chelate the metal ion with the ELP-chelator, thereby preparing an ELP contrast enhancement agent.

54. The method of claim 53, wherein the bifunctional chelator group forms a covalent bond with the amine group on the ELP.

55. The method of claim 54, wherein the covalent bond is one of an amide and a thiourea.
